(19) United States
(12) Patent Application Publication
(10) Pub. No.: US 2002/0128191 A1
(43) Pub. Date: Sep. 12, 2002

(54) Methods of treating conditions associated with corneal injury

(75) Inventor: Patrick J. Scannon, San Francisco, CA (US)

Correspondence Address: Janet M. McNicholas, Ph.D., McAndrews, Held & Malloy, Ltd., 34th Floor, 500 W. Madison Street, Chicago, IL, 60661, US

(73) Assignee: XOMA Corporation

(21) Appl. No.: 09/941,198

(22) Filed: Aug. 27, 2001

Related U.S. Application Data
(63) Continuation of application No. 09/677,507, filed on Oct. 2, 2000.
Application No. 09/677,507 is a continuation of application No. 09/189,040, filed on Nov. 10, 1998, now abandoned.
Application No. 09/189,040 is a continuation of application No. 08/557,289, filed on Nov. 14, 1995, now abandoned.

(30) Foreign Application Priority Data
Nov. 12, 1996 (US) PCT/US96/18632

(51) Int. Cl. A61K 38/17

(52) U.S. Cl. 514/12

(57) ABSTRACT

The present invention provides methods of treating a subject suffering from adverse effects, complications or conditions, associated with or resulting from a corneal injury including, corneal infection or ulceration, by topical administration of suitable ophthalmic preparations of bactericidal/permeability-increasing (BPI) protein products.

### BACKGROUND OF THE INVENTION

The present invention elates generally to methods of treating a subject suffering from adverse effects, complications or conditions including infection or ulceration associated with or resulting from corneal injury from, for example, perforation, abrasion, chemical bum or trauma injury, by topical administration of bactricidal/permeability-increasing (BPI) protein products.

Corneal infections, microbial keratitis and infectious corneal ulceration are increasingly prevalent, serious and sight-threatening ophthalmic diseases. Infectious or microbial keratitis is an infection of the cornea characterized by an ulceration of the corneal epithelium associated with an underlying inflammatory infiltrate of the corneal stroma. Infectious keratitis is the most serious complication of wearing contact lenses. Complications of infectious keratitis include sight-thing scar formation, scleral involvement, corneal perforation, and even loss of the eye. Corneal diseases are estimated to involve several hundred thousand cases of corneal ulcers and about twice that number of keratitis cases each year in the U.S. alone. Contact lens wearers, immunocompromised individuals and patients suffering from dry eye syndrome are among those most at risk to develop such corneal lesions. In third world countries, this cause of blindness is second only to cataract formation.

Microbial keratitis, or infections of the cornea, can be caused by various bacteria, fungi, viruses, or parasites. Bacteria are the most common causes, but the frequency of involvement of different species may vary from one geographic region to another and may show a shifting pattern over time. Species of bacteria causing keratitis in the majority of cases are: (1) Micrococcaceae (Staphylococcus, Micrococcus), (2) Streptococci, (3) Pseudomonas, and (4) Enterobacteriaceae (Citrobacter, Klebsiella, Enterobacter, Serratia, Proteus). Historically, the pneumococcus (Streptococcus pneumoniae) was a major cause, but now other gram-positive organisms predominate, with Staphylococcus aureus reported to be the most common cause of microbial keratitis in the northern United States. Pseudomonas aeruginosa has also become more prevalent as a cause of keratitis, particularly in association with overnight contact lens wear. Infections involving the indigenous bacteria of the conjunctiva and eyelids (Staphylococcus epidermidis, Corynebacterium and Propionibacterium species) are reportedly being seen more frequently, as are other commensal and less virulent organisms, especially in immunocompromised hosts. The variety of organisms most commonly seen in bacterial keratitis has been documented (see, e.g., Liesegang, Bacterial Keratitis, in Infectious Disease Clinics of North America, Vol. 6, No. 4, pp.815-829, December, 1992); however, any organism, under appropriate circumstances, can be a causative agent of corneal infection and ulceration.

Corneal infection is usually precipitated by an epithelial defect resulting from injury (including perforation, abrasion, chemical burn or trauma injury) to the cornea or from contact lens wear. Corneal disease patients and patients receiving topical corticosteroids or with compromised local or systemic defense mechanisms appear more susceptible to corneal epithelial defects precipitating infection.

The cornea is an avascular structure, and has a protective coating with two layers of mucosubstances, including an adherent glycocalyx and a mucin layer produced by goblet cells. The intact corneal epithelium is usually an effective barrier against infection, although some bacterial organisms, notably Neisseria gonorrhoeae and Corynebacterium diphtheriae, can penetrate the intact epithelium.

The lids and eyelashes normally harbor microorganisms and shed them onto the cornea, but the eyelids provide a defensive system for the cornea, primarily through the lacrimal secretions and the ocular blink reflex.

The tear film provides lubrication to flush away any organisms or debris. The tear film also contains several antimicrobial substances, including lysozyme, lactoferrin, beta-lysins, and complement components, as well as immunoglobulins (especially secretory IgA) and lymphocytes, which provide a local defense mechanism. Lactoferrin can enhance the effect of surface antibodies or inhibit bacterial growth or invasiveness by chelating iron. Tear lysozyme can directly lyse bacterial cell walls, and beta-lysins can lyse bacterial membranes. Secretory IgA blocks the adhesion of bacteria to membranes. Malposition of the lids and lashes, however, or difficulty in lid closure interferes with these protective functions and predisposes to corneal infection.

Predisposing factors to corneal infection therefore include: (1) trauma or injury (e.g., foreign body, contact lens wear); (2) abnormal tear function (e.g., dry eye, lacrimal obstruction) and abnormal lid structure and function (e.g., blepharitis, laopthalmus entropion, ectropion, trichiasis); (3) comeal diseases (e.g., corneal edema); and (4) systemic conditions (e.g., Sjögren's syndrome, alcoholism, diabetes, rheumatoid arthritis, debilitating disease, tracheal intubation, central nervous system disease and psychiatric disturbances, extensive bums, acquired immunodeficiency syndrome (AIDS), and corticosteroid and immunosuppressive therapy).

Contact lens wear is a significant risk factor compromising the structural integrity of the corneal epithelium and predisposing toward corneal infection. Contact lens wear give rise to corneal hypoxia, increased corneal temperature, decreased tear flow to the cornea, and also provides a constant source of microtrauma to the corneal epithelium. Soft contact lenses become coated with mucus and protein after only a few hours of wear, and this may further enhance the adherence of bacteria. Hard gas-permeable lenses, daily wear soft contact lenses, extended wear soft contact lenses, therapeutic soft contact lenses, and disposable contact lenses all increase the risk of microbial keratitis. Overnight wear, especially after cataract surgery, is associated with the highest risk. Other factors contributing to contact lens-associated microbial keratitis include the failure to follow proper contact lens wear instructions, poor contact lens hygiene, use of contaminated lens solutions, and microtrauma at the time of the insertion and removal. Pseudomonas aeruginosa and Staphylococcus are the most common organisms isolated in contact lens-associated keratitis.

Acanthamoeba keratitis, a parasitic infection, has been linked to prolonged exposure to contaminated water, especially in contact lens wearers and in individuals who use hot tubs or swimming pools. Fungal keratitis is seen in different clinical situations. Filamentary fungal keratitis is seen after injury to the cornea in agricultural settings, whereas yeast keratitis is seen in any environment in patients who are immunocompromised or have a severely damaged cornea.

The severity of the bacterial keratitis depends, for the most part, on the virulence of the invading bacteria but also is correlated to the previous health of the cornea and the host response. The pathogenicity of particular organisms is correlated with the ability to adhere to the edge or base of an epithelial defect and to invade the corneal stroma. Pseudomonas aeruginosa, Staphylococcus aureus, and Streptococcus pneumoniae adhere tightly to the edge of an epithelial defects, probably because of membrane appendages called fibrillae (in gram-positive organisms) or fimbriae (in gram-negative organisms). Specific adhesions on the surface of these appendages may interact with specific receptors on the corneal epithelium. Some species, notably Pseudomonas and Staphylococcus, produce an extracellular polysaccharide slime layer which may have a role in adherence to a variety of surfaces, especially soft contact lenses. The mechanisms of penetration of bacteria into the corneal stroma following entry through an epithelial injury are poorly understood but are probably correlated with the production of toxins and enzymes. Pseudomonas and Serratia species have proteoglycanase (e.g., collagenase) activity that can liquify the stroma. Other organisms have other properties that permit adherence and corneal destruction. The host's polymorphonuclear response to the infection contributes to the tissue destruction and collagen breakdown as a result of lysozymal enzymes and other proteases.

In a previously healthy cornea, the presence of a corneal epithelial ulceration with adherent mucopurulent exudate and inflammatory cells in the adjacent corneal stroma and the anterior chamber should lead to a presumptive diagnosis of bacterial keratitis. The eyelids may be stuck together and the tear film filled with inflammatory cells. Nonspecific symptoms include decreased vision, redness, pain, conjunctival and lid swelling and a discharge. Clinical signs may include increasing stromal edema, hypopyon, iris miosis, and synechiae.

In a patient with a cornea previously damaged by herpes simplex virus infection, corneal edema, or trauma, it may be difficult to distinguish the clinical signs of infection from the residua of the underlying structural abnormalities. A bacterial infection should be suspected when there is an increase in the extent of epithelial or stromal ulceration or anterior chamber inflammation. Antecedent therapy with systemic or local ocular immunosuppressive agents, especially corticosteroids, not only increases the risk of ocular infection but may alter the clinical response in such a way as to mask or alter some of the typical features of infection.

There are difficulties in distinguishing bacterial keratitis from other forms of microbial keratitis or from the multiple noninfectious causes of corneal ulceration. The differential diagnosis includes fungal, viral, and parasitic keratitis as well as toxic or chemical keratopathy, indolent or neurotrophic ulceration, severe dry eyes, and various other insults to the cornea. The history, physical examination, and evidence of the onset of the new disease process may permit a presumptive diagnosis. When corneal infection is suspected, the culture strategy may include screening for the most likely agents: aerobic bacteria, anaerobic bacteria, filamentous fungi, and yeasts. A corneal sample may be obtained by scraping, using the magnification of the slit lamp biomicroscope, and topical anesthesia. With deep keratitis, fragments of the cornea may be excised with a microsurgical scissor or trephine. More than one species of microbe may be present in a corneal infection. Negative cultures are not uncommon in cases of suspected infectious corneal ulcers, and may be due to inadequate sampling methods, the improper selection of media, prior antibiotic treatment, or improper interpretation of data.

Currently, the initial therapy for suspected microbial keratitis is based on the severity of the keratitis and a familiarity with the most likely causative organisms. Suspected microbial keratitis is typically treated as a bacterial ulcer until a more definitive laboratory diagnosis is made. Initial antibiotic therapy may be based on the results of the Gram stain or Giemsa stain, or a broad spectrum antibiotic may be administered as the initial treatment, especially in cases of serious suspected microbial keratitis. Most U.S. practitioners are not willing to leave the lesion untreated while waiting for culture results. Generally, a broad spectrum antibiotic is prescribed following examination. Such initial antibiotic therapy may be modified after the causative organism is identified from correlation of the Gram stain, culture results, and the clinical response. There are a relatively small number of antibiotics available commercially as topical ophthalmic preparations. Many other antibiotics can be prepared for topical ophthalmic use in treating serious corneal infections, however, their use is expensive and inconvenient, and many are not well tolerated or have limited antibacterial spectra. Pseudomonas species account for many serious, and rapidly destructive, corneal infections. In fact, ocular disease produced by the opportunistic bacterial pathogen P. aeruginosa often leads to a fulminating and highly destructive infection resulting in rapid liquefaction of the cornea and blindness. Antibiotic treatment is not always successful due to the resistance of many clinical strains. The patient is vulnerable during the ulcerative period to sequelae that are sight threatening and even could create a situation where the eye had to be enucleated. Any agent that could accelerate the healing time, for example, would be highly desired by medical practitioners. Thus, there is an unmet need to develop agents with therapeutic efficacy, either alone or in conjunction with existing agents, against these organisms.

In cases where there is the need for frequent administration of antimicrobial drops and the need to examine the patient daily, patients may be hospitalized. Patient isolation is not usually necessary, although contact with preoperative patients should be avoided. Outpatient therapy may be preferred for compliant patients or those with milder disease.

The ideal topical antibiotic agent should be bactericidal at reasonable concentrations against the corneal pathogens, should be able to penetrate the cornea, and should be free of significant adverse affects. Factors considered in the use of systemic antibiotics (i.e., achievable serum levels, distribution space, and absorption and excretion characteristics) are not applicable. Some patients may respond to commercial-strength topical antibiotic agents given at frequent intervals, but fortified topical antibiotic agents are usually more effective. For example, recent fluoroquinolone antibiotics, norfloxacin and ciprofloxacin, may be effective at commercial strength for infections by susceptible bacteria. Drug penetration into the cornea may be increased with higher concentration of the drug, more frequent application, longer contact time with the use of some vehicles, with more lipophilic antibiotic agents, and with the absence of the epithelium. Solutions may be preferred to ointments because of the flexibility in varying the concentration and the ease of administration. A fortified topical antibiotic agent may be prepared by adding the desired amount of the parenteral antibiotic to an artificial tear solution.

The primary goal of current therapy is to administer an antibiotic which will be effective quickly without causing significant ocular and systemic toxicity. Other considerations or goals are to reduce the corneal inflammatory response, to limit structural corneal damage, and to promote corneal reepithelialization. As is the case in other organ systems, healing of a corneal ulcer is often accompanied by neovascularization. In the eye, neovascularization and scarring are particularly deleterious as vision is dependent upon a clear cornea which requires the maintenance of the highly organized fibrin structure. Immunosuppressant corticosteroids can be used to inhibit the vessel formation but many ophthalmologists would rather not risk this indiscriminate type of immune suppression while the cornea is vulnerable due to ulceration. Thus, there exists a need in the art for agents with therapeutic efficacy in reduction of neovascula ization and scanning but without the generalized immune suppressing effects of steroids.

Even with current antibiotic and steroid therapies, major concerns regarding the treatment of infectious corneal ulcers remain, including: broad spectrum application; fear of antibiotic resistant strains of microbes; controversy regarding prophylactic versus therapeutic treatment of suspected infectious ulcers; non-compliant patients; control of neovascularization and scar formation. There exists a need for new therapeutic agents that would better address these issues.

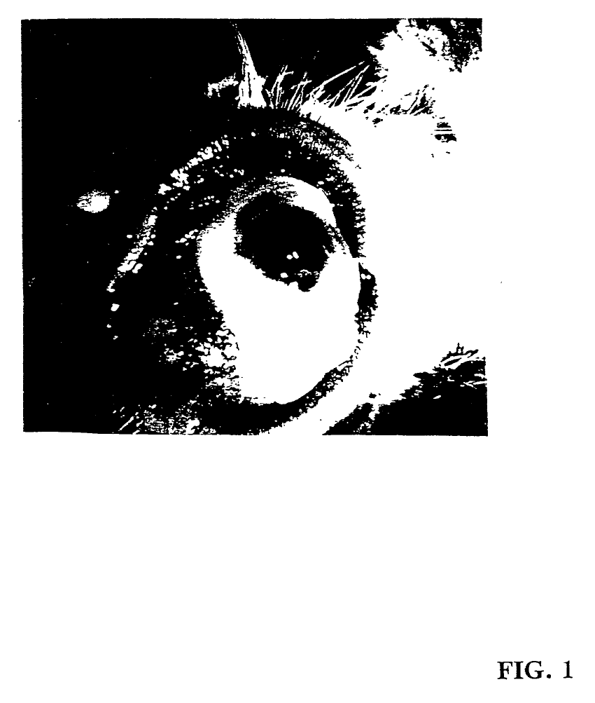

BPI is a protein isolated from the granules of mammalian polymorphonuclear leukocytes (PMNs or neutrophils), which are blood cells essential in the defense against invading microorganisms. Human BPI protein has been isolated from PMNs by acid extraction combined with either ion exchange chromatography [Elsbach, J. Biol. Chem., 254:11000 (1979)] or E. coli affinity chromatography [Weiss, et al., Blood, 69:652 (1987)]. BPI obtained in such a manner is referred to herein as natural BPI and has been shown to have potent bactericidal activity against a broad spectrum of gram-negative bacteria. The molecular weight of human BPI is approximately 55,000 daltons (55 kD). The amino acid sequence of the entire human BPI protein and the nucleic acid sequence of DNA encoding the protein have been reported in FIG. 1 of Gray et al., J. Biol. Chem., 264:9505 (1989), incorporated herein by reference. The Gray et al. amino acid sequence is set out in SEQ ID NO: 1 hereto.

BPI is a strongly cationic protein. The N-terminal half of BPI accounts for the high net positive charge; the C-terminal half of the molecule has a net charge of -3. [Elsbach and Weiss (1981), supra.] A proteolytic N-terminal fragment of BPI having a molecular weight of about 25 kD has an amphipathic character, containing alternating hydrophobic and hydrophilic regions. This N-terminal fragment of human BPI possesses the anti-bacterial efficacy of the naturally-derived 55 kD human BPI holoprotein. [Ooi et al., J. Bio. Chem., 262: 14891-14894 (1987)]. In contrast to the N-terminal portion, the C-terminal region of the isolated human BPI protein displays only slightly detectable anti-bacterial activity against gram-negative organisms. [Ooi et al., J. Exp. Med., 174:649 (1991).] An N-terminal BPI fragment of approximately 23 kD, referred to as “rBPI₂₃,” has been produced by recombinant means and also retains anti-bacterial activity against gram-negative organisms. Gazzano-Santoro et al., Infect. Immun. 60:4754-4761 (1992).

There continues to exist a need in the art for new methods and materials for treatment of corneal injury, including infection or ulceration. Products and methods responsive to this need would ideally involve substantially non-toxic, non-irritating ophthalmic preparations available in suitable amounts by means of synthetic or recombinant methods. Ideal compounds would be capable of penetrating corneal tissue and would prevent or reduce the number and severity of adverse effects, complications or conditions associated with or resulting from corneal injury. Alternatively, or in addition, such ideal compounds would enhance the effect of, or reduce the need for, other concurrently administered anti-inflammatory and/or antimicrobial therapeutic agents.

### SUMMARY OF THE INVENTION

The present invention provides novel methods of treating corneal epithelial injury associated infection comprising topical application to the cornea of a subject having a corneal epithelial injury a bactericidal/permeability-increasing (BPI) protein product in an amount effective to reduce hyperemia, chemosis, neovascularization, mucous discharge or ulcer formation. Methods according to the invention are thus useful for reducing the adverse effects, complications or conditions associated with or resulting from a corneal injury including, corneal infection or ulceration, by topically administering a therapeutically effective amount of an ophthalmic preparation of a BPI protein product to a subject suffering from the effects of such corneal infection, ulceration or injury. The invention derives in part from the surprising discovery that topically administered BPI protein products penetrate the cornea and prevent or reduce adverse effects associated with corneal infections and ulcerations. These adverse effects include hyperemia, chemosis, mucous discharge, tearing, photophobia, keratitis, neovascularization, ulcer formation, opacification (clouding), contrast sensitivity, scarring, pain or loss of visual acuity. Confirmation of beneficial effects of practice of the invention is provided by standard ophthalmological examination including, for example, slit lamp biomicroscopy.

Methods of the present invention contemplate administration of a BPI protein product in ophthalmologically acceptable preparations which may include, or be concurrently administered with, anti-inflammatory agents such as corticosteroids and/or antimicrobial agents such as ciprofloxacin gentamicin, ofloxacin and anti-fungal agents. Presently preferred BPI protein products of the invention include biologically active amino terminal fragments of the BPI holoprotein, recombinant products such as rBPI₂₁ and rBPI₄₂ and recombinant or chemically synthesized BPI-derived peptides as described in detail below.

The invention further provides for the use of a BPI protein products for manufacture of a topical medicament for reducing the above-noted adverse effects, complications or conditions, associated with or resulting from corneal infection and ulceration.

Numerous additional aspects and advantages of the invention will become apparent to those skilled in the art upon considering the following detailed description of the invention, which describes the presently preferred embodiments thereof, reference being made to the drawing wherein:

FIG. 1 is a photograph of a “control” rabbit eye 72 hours after corneal epithelium puncture and injection with Pseudomonas aeruginosa wherein post-injection treatments included an ophthalmic product vehicle solution only; and

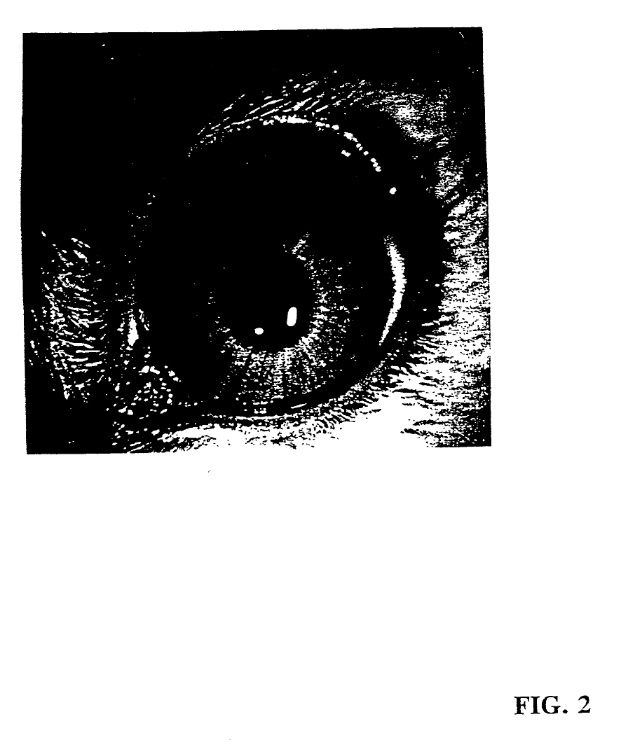

FIG. 2 is a photograph of a rabbit eye 72 hours after corneal epithelium puncture and injection with Pseudomonas aeruginosa wherein the cornea was treated according to the present invention.

### DETAILED DESCRIPTION OF THE INVENTION

Incorporated by reference herein are the disclosures of the applicant's co-owned, co-pending, concurrently-filed U.S. patent application Ser. No. ______ (Attorney Docket No. 27129/33007) entitled “Methods of Treating Conditions Associated With Corneal Transplantation.”

The present invention relates to the surprising discovery that a bactericidal/permeability-increasing (BPI) protein product can be topically administered to the cornea, in an amount effective to reduce hyperemia, chemosis, neovascularization, mucous discharge or ulcer formation associated with or resulting from corneal epithelial injury associated infection. Methods according to the invention are useful for treating subjects suffering from corneal infection, ulceration, or injury, and conditions associated therewith or resulting therefrom. Particularly valuable is the lack of corneal tissue toxicity and the effectiveness of such topically administered BPI protein products, given that penetration of corneal tissue is a necessary but not sufficient step for therapeutic efficacy. BPI protein products are shown herein to prevent or reduce adverse effects of corneal injury associated infection and ulceration including, for example, preventing or reducing hyperemia, chemosis, mucous discharge, tearing, photophobia, keratitis, neovascularization, ulcer formation (i.e., prevent ulcer development or reduce ulcer size) opacification (clouding), contrast sensitivity, scanning, pain and loss of visual acuity as measured by standard ophthalmological examination, using, slit lamp biomicroscopy to note clinical manifestations.

According to one aspect of the invention, suitable ophthalmic preparations of BPI protein product alone, in an amount sufficient for monotherapeutic effectiveness, may be administered to a subject suffering from corneal infection, ulceration, or injury, and conditions associated therewith or resulting therefrom. When used to describe administration of BPI protein product alone, the term “amount sufficient for monotherapeutic effectiveness” means a suitable ophthalmic preparation having an amount of BPI protein product that provides beneficial effects, including anti-microbial and/or anti-angiogenic effects, when administered as a monotherapy. The invention utilizes any of the large variety of BPI protein products known to the art including natural BPI protein isolates, recombinant BPI protein, BPI fragments, BPI analogs, BPI variants, and BPI-derived peptides.

According to another aspect of the invention, a patient may be treated by concurrent administration of suitable ophthalmic preparations of a BPI protein product in an amount sufficient for combinative therapeutic effectiveness and one or more immunosuppressant corticosteroids in amounts sufficient for combinative therapeutic effectiveness. This aspect of the invention contemplates concurrent administration of BPI protein product with any corticosteroid or combinations of corticosteroids, including prednisolone and dexamethasone and contemplates that, where corticosteroid therapy is required, lesser amounts will be needed and/or that there will be a reduction in the duration of treatment.

According to another aspect of the invention, a subject suffering from corneal epithelial injury associated infection or ulceration, and conditions associated therewith or resulting therefrom, may be treated by concurrent administration of suitable ophthalmic preparations of a BPI protein product in an amount sufficient for combinative therapeutic effectiveness and one or more antibiotics in amounts sufficient for combinative therapeutic effectiveness. This aspect of the invention contemplates concurrent administration of BPI protein product with any antimicrobial agent or combinations thereof for topical use in the eye including: antibacterial agents such as gentamicin, tobramycin, bacitracin, chloramphenicol, ciprofloxacin, ofloxacin, norfloxacin, erythromycin, bacitracin/neomycin/polymyxin B, sulfisoxazole, sulfacetamide, tetracycline, polymyxin/bacitracin, trimethroprim/polymyxin B, vancomycin, clindamycin, ticarcillin, penicillin, oxillin or cefazolin; antifungal agents such as amphotericin B, nystatin, natamycin (pimaricin), miconazole, ketocanozole or fluconazole; antiviral agents such as idoxuridine, vidarabine or trifluridine; and antiprotozoal agents such as propamidine, neomycin, clotrimazol, miconazole, itraconazole or polyhexamethylene biguanide.

This aspect of the invention is based on the improved therapeutic effectiveness of suitable ophthalmic preparations of BPI protein products with antibiotics, e.g., by increasing the antibiotic susceptibility of infecting organisms to a reduced dosage of antibiotics providing benefits in reduction of cost of antibiotic therapy and/or reduction of risk of toxic responses to antibiotics. BPI protein products may lower the minimum concentration of antibiotics needed to inhibit in vitro growth of organisms at 24 hours. In cases where BPI protein product does not affect growth at 24 hours, BPI protein product may potentiate the early bactericidal effect of antibiotics in vitro at 0-7 hours. The BPI protein products may exert these effects even on organisms that are not susceptible to the direct bactericidal or growth inhibitory effects of BPI protein product alone.

This aspect of the invention is correlated to effective reversal of the antibiotic resistance of an organism by administration of a BPI protein product and antibiotic. BPI protein products may reduce the minimum inhibitory concentration of antibiotics from a level within the clinically resistant range to a level within the clinically susceptible range. BPI protein products thus may convert normally antibiotic-resistant organisms into antibiotic-susceptible organisms.

According to these aspects of the invention, suitable ophthalmic preparations of the BPI protein product along with corticosteroids and/or antibiotics are concurrently administered in amounts sufficient for combinative therapeutic effectiveness. When used to describe administration of a suitable ophthalmic preparation of BPI protein product in conjunction with a corticosteroid, the term “amount sufficient for combinative therapeutic effectiveness” with respect to the BPI protein product means at least an amount effective to reduce or minimize neovasculaization and the term “amount sufficient for combinative therapeutic effectiveness” with respect to a corticosteroid means at least an amount of the corticosteroid that reduces or minimizes inflammation when administered in conjunction with that amount of BPI protein product. Either the BPI protein product or the corticosteroid, or both, may be administered in an amount below the level required for monotherapeutic effectiveness against adverse effects associated with or resulting from corneal injury associated infection/ulceration. When used to describe administration of a suitable ophthalmic preparation of BPI protein product in conjunction with an antimicrobial, the term “amount sufficient for combinative therapeutic effectiveness” with respect to the BPI protein product means at least an amount effective to reduce neovascularization and/or increase the susceptibility of the organism to the antimicrobial, and the term “amount sufficient for combinative therapeutic effectiveness” with respect to an antimicrobial means at least an amount of the antimicrobial that produces bactericidal or growth inhibitory effects when administered in conjunction with that amount of BPI protein product. Either the BPI protein product or the antimicrobial, or both, may be administered in an amount below the level required for monotherapeutic effectiveness.

BPI protein product may be administered in addition to standard therapy and is preferably incorporated into the care given the patient exposed to risk of corneal epithelium injury or actually suffering such injury. Treatment with BPI protein product is preferably continued for at least 1 to 30 days, and potentially longer if necessary, in dosage amounts (e.g., dropwise administration of about 10 to about 200 μL solution of a BPI protein product at about 1 to 2 mg/mL) determined by good medical practice based on the clinical condition of the individual patient.

Suitable ophthalmic preparations of BPI protein products may provide benefits as a result of their ability to neutralize heparin and their ability to inhibit heparin-dependent angiogenesis. The anti-angiogenic properties of BPI have been described in Little et al., co-owned, co-pending U.S. application Ser. No. 08/435,855 and co-owned U.S. Pat. No. 5,348,942, both incorporated by reference herein.

Suitable ophthalmic preparations of BPI protein products may provide additional benefits as a result of their ability to neutralize endotoxin associated with gram-negative bacteria and/or endotoxin released by antibiotic treatment of patients with corneal infection/ulceration. Suitable ophthalmic preparations of BPI protein products could provide further benefits due to their anti-bacterial activity against susceptible bacteria and fungi, and their ability to enhance the therapeutic effectiveness of antibiotics and anti-fungal agents. See, e.g., Horwitz et al., co-owned, co-pending U.S. application Ser. No. 08/372,783, filed Jan. 13, 1995 as a continuation-in-part of U.S. application Ser. No. 08/274,299, filed Jul. 11, 1994, which are all incorporated herein by reference and which describe BPI protein product activity in relation to gram-positive bacteria; and Little et al., co-owned, co-pending U.S. application Ser. No. 08/372,105, filed Jan. 13, 1995 as a continuation-in-part of U.S. application Ser. No. 081273,540, filed Jul. 11, 1994, which are all incorporated herein by reference and which describe BPI protein product activity in relation to fungi.

For ophthalmic uses as described herein, the BPI protein product is preferably administered topically, to the corneal wound or injury. Topical routes include administration preferably in the form of ophthalmic drops, ointments, gels or salves. Other topical routes include irrigation fluids (for, e.g., irrigation of wounds). Those skilled in the art can readily optimize effective ophthalmic dosages and administration regimens for the BPI protein products.

As used herein, “BPI protein product” includes naturally and recombinantly produced BPI protein; natural, synthetic, and recombinant biologically active polypeptide fragments of BPI protein; biologically active polypeptide variants of BPI protein or fragments thereof, including hybrid fusion proteins and dimers; biologically active polypeptide analogs of BPI protein or fragments or variants thereof, including cysteine-substituted analogs; and BPI-derived peptides. The BPI protein products administered according to this invention may be generated and/or isolated by any means known in the art. U.S. Pat. No. 5,198,541, the disclosure of which is incorporated herein by reference, discloses recombinant genes encoding and methods for expression of BPI proteins including recombinant BPI holoprotein, referred to as rBPI₅₀ or rBPI₅₅ and recombinant fragments of BPI. Co-owned, copending U.S. patent application Ser. No. 07/885,501 and a continuation-in-part thereof, U.S. patent application Ser. No. 08/072,063 filed May 19, 1993 and corresponding PCT Application No. 93/04752 filed May 19, 1993, which are all incorporated herein by reference, disclose novel methods for the purification of recombinant BPI protein products expressed in and secreted from genetically transformed mammalian host cells in culture and discloses how one may produce large quantities of recombinant BPI products suitable for incorporation into stable, homogeneous pharmaceutical preparations.

Biologically active fragments of BPI (BPI fragments) include biologically active molecules that have the same or similar amino acid sequence as a natural human BPI holoprotein, except that the fragment molecule lacks amino-terminal amino acids, internal amino acids, and/or carboxy-terminal amino acids of the holoprotein. Nonlimiting examples of such fragments include a N-terminal fragment of natural human BPI of approximately 25 kD, described in Ooi et al., J. Exp. Med., 174:649 (1991), and the recombinant expression product of DNA encoding N-terminal amino acids from 1 to about 193 or 199 of natural human BPI, described in Gazzano-Santoro et al., Infect. Immun. 60:4754-4761 (1992), and referred to as rBPI₂₃. In that publication, an expression vector was used as a source of DNA encoding a recombinant expression product (rBPI₂₃) having the 31-residue signal sequence and the first 199 amino acids of the N-terminus of the mature human BPI, as set out in FIG. 1 of Gray et al., supra, except that valine at position 151 is specified by GTG rather than GTC and residue 185 is glutamic acid (specified by GAG) rather than lysine (specified by AAG). Recombinant holoprotein (rBPI) has also been produced having the sequence (SEQ ID NOS: 1 and 2) set out in FIG. 1 of Gray et al., supra, with the exceptions noted for rBPI₂₃ and with the exception that residue 417 is alanine (specified by GCT) rather than valine (specified by GTI). Other examples include dimeric forms of BPI fragments, as described in co-owned and co-pending U.S. Pat. No. 5,447,913 the disclosures of which are incorporated herein by reference. Preferred dimeric products include dimeric BPI protein products wherein the monomers are amino-terminal BPI fragments having the N-terminal residues from about 1 to 175 to about 1 to 199 of BPI holoprotein. A particularly preferred dimeric product is the dimeric form of the BPI fragment having N-terminal residues 1 through 193, designated rBPI₄₂ dimer.

Biologically active variants of BPI (BPI variants) include but are not limited to recombinant hybrid fusion proteins, comprising BPI holoprotein or biologically active fragment thereof and at least a portion of at least one other polypeptide, and dimeric forms of BPI variants. Examples of such hybrid fusion proteins and dimeric forms are described by Theofan et al. in co-owned, copending U.S. patent application Ser. No. 07/885,911, and a continuation-in-part application thereof, U.S. patent application Seri. No. 08/064,693 filed May 19, 1993 and corresponding PCT Application No. US93/04754 filed May 19, 1993, which are all incorporated herein by reference and include hybrid fusion proteins comprising, at the amino-terminal end, a BPI protein or a biologically active fragment thereof and, at the carboxy-terminal end, at least one constant domain of an immunoglobulin heavy chain or allelic variant thereof.

Biologically active analogs of BPI (BPI analogs) include but are not limited to BPI protein products wherein one or more amino acid residues have been replaced by a different amino acid. For example, co-owned, U.S. Pat. No. 5,420,019 and corresponding PCT Application No. US94/01235 filed Feb. 2, 1994, the disclosures of which are incorporated herein by reference, discloses polypeptide analogs of BPI and BPI fragments wherein a cysteine residue is replaced by a different amino acid. A preferred BPI protein product described by this application is the expression product of DNA encoding from amino acid 1 to approximately 193 (particularly preferred) or 199 of the N-terminal amino acids of BPI holoprotein, but wherein the cysteine at residue number 132 is substituted with alanine and is designated rBPI₂₁Δcys or rBPI₂₁. Other examples include dimeric forms of BPI analogs; e.g. co-owned and co-pending U.S. patent application Ser. No. 08/212,132 filed Mar. 11, 1994, the disclosures of which are incorporated herein by reference.

Other BPI protein products useful according to the methods of the invention are peptides derived from or based on BPI produced by synthetic or recombinant means (BPI-derived peptides), such as those described in PCT Application No. US95/09262 filed Jul. 20, 1995 corresponding to co-owned and copending U.S. application Ser. No. 08/504,841 filed Jul. 20, 1995, PCT Application No. US94/10427 filed Sep. 15, 1994, which corresponds to U.S. patent application Ser. No. 08/306,473 filed Sep. 15, 1994, and PCT Application No. US94/02465 filed Mar. 11, 1994, which corresponds to U.S. patent application Ser. No. 08/209,762, filed Mar. 11, 1994, which is a continuation-in-part of U.S. patent application Ser. No. 08/183,222, filed Jan. 14, 1994, which is a continuation-in-part of U.S. patent application Ser. No. 08/093,202 filed Jul. 15, 1993 (for which the corresponding international application is PCT Application No. US94/02401 filed Mar. 11, 1994), which is a continuation-in-part of U.S. patent application Ser. No. 08/030,644 filed Mar. 12, 1993, the disclosures of all of which are incorporated herein by reference.

The safety of BPI protein products for systemic administration to humans has been established healthy volunteers and in human experimental endotoxemia studies published in von der Möhlen et al., Blood, 85(12):3437-3343 (1995) and von der Möhlen et al., J. Infect. Dis., 172:144-151 (1995).

Presently preferred BPI protein products include recombinantly-produced N-terminal fragments of BPI, especially those having a molecular weight of approximately between 21 to 25 kD such as rBPI₂₁ or rBPI₂₃; or dimeric forms of these N-terminal fragments (e.g., rBPI₄₂ dimer). Additionally, preferred BPI protein products include rBPI55 and BPI-derived peptides. Presently most preferred is the rBPI₂₁ protein product.

The administration of BPI protein products is preferably accomplished with a pharmaceutical composition comprising a BPI protein product and a pharmaceutically acceptable diluent, adjuvant, or carrier. The BPI protein product may be administered without or in conjunction with known surfactants, other chemotherapeutic agents or additional known antimicrobial agents. Presently preferred pharmaceutical compositions containing BPI protein products (i.e., rBPI₂₁) comprise the BPI protein product at a concentration of 2 mg/ml in 5 mM citrate, 150 mM NaCl, 0.2% poloxamer 403 (Pluronic P123, BASF Wyandotte, Parsippany, N.J.) (most preferred) or 0.2% poloxamer 333 (Pluronic P103 BASF Wyandotte, Parsippany, N.J.) and 0.002% polysorbate 80 (Tween 80, ICI Americas Inc., Wilmington, Del.). Compositions of BPI protein product and anti-bacterial activity-enhancing poloxamer surfactants are described in co-owned, co-pending U.S. patent application Ser. No. 08/372,104 filed Jan. 13, 1995 and Ser. No. 08/530,599 filed Sep. 19, 1995 the disclosures of which are incorporated herein by reference. Another pharmaceutical composition containing BPI protein products (i.e., rBPI₂₁) comprises the BPI protein product at a concentration of 2 mg/ml in 5 mM citrate, 150 mM NaCl, 0.2% poloxamer 188 (Pluronic F 68, BASF Wyandotte, Parsippany, N.J.) and 0.002% polysorbate 80. Yet another pharmaceutical composition containing BPI protein products (e.g., rBPI₅₅, rBPI₄₂, rBPI23) comprises the BPI protein product at a concentration of 1 mg/ml in citrate buffered saline (5 or 20 mM citrate, 150 mM NaCl, pH 5.0) comprising 0.1% by weight of poloxamer 188 (Pluronic F-68, BASF Wyandotte, Parsippany, N.J.) and 0.002% by weight of polysorbate 80 (Tween 80, ICI Americas Inc., Wilmington, Del.). Such combinations are described in co-owned, co-pending PCT Application No. US94/01239 filed Feb. 2, 1994, which corresponds to U.S. patent application Ser. No. 08/190,869 filed Feb. 2, 1994 and U.S. patent application Ser. No. 08/012,360 filed Feb. 2, 1993, the disclosures of all of which are incorporated herein by reference.

Other aspects and advantages of the present invention will be apparent upon consideration of the following illustrative examples wherein: Example 1 addresses the effects of various BPI protein products with respect to Pseudomonas infection in a corneal infection/ulceration rabbit model; Example 2 addresses the effects of varying formulations of a single BPI protein product with respect to Pseudomonas infection in a corneal infection/ulceration rabbit model; Example 3 addresses the effects of BPI protein product administration on Pseudomonas infection in a corneal infection/ulceration rabbit model either alone and in co-administration with various antibiotics.

### EXAMPLE 1

### Effect pf BPI Protein Products on Pseudomonas Infection in a Corneal Ulceration Rabbit Model

The effects of various BPI protein products were first evaluated in the context of administration both prior to and after Pseudomonas infection m a corneal infection/ulceration rabbit model. BPI protein products tested included: rBPI42 (Expt. 1), rBPI₂₁ in a formulation with poloxamer 188 (Expt. 2), an anti-angiogenic BPI-derived peptide designated XMP.112 (Expt. 3), an anti-bacterial BPI-derived peptide designated XMP.105 (Expt. 4) and rBPI₂₁ in a formulation with poloxamer 403 (Expt. 5). The structure of XMP.112 and XMP.105 are set out in previously-noted PCT Application No. 94/02465.

For these experiments, the infectious organism was a strain of Pseudomonas aeruginosa 19660 obtained from the American Type Culture Collection (ATCC, Rockville, Md.). The freeze dried organism was resuspended in nutrient broth (Difco, Detroit, Mich.) and grown at 37° C. with shaking for 18 hours. The culture was centrifuged following the incubation in order to harvest and wash the pellet. The washed organism was Gram stained in order to confirm purity of the culture. A second generation was cultured using the same techniques as described above. Second generation cell suspensions were diluted in nutrient broth and adjusted to an absorbance of 1.524 at 600 nm, a concentration of approximately 6.55×10⁹ CFU/ml. A final 1.3×10⁶ fold dilution in nutrient broth yielded 5000 CFU/mL or 1.0×10² CFU/0.02 mL. Plate counts for CFU determinations were made by applying 100 μL of the diluted cell suspension to nutrient agar plates and incubating them for 24-48 hours at 37° C.

For these experiments, the animals used were New Zealand White rabbits, maintained in rigid accordance to both SERI guidelines and the ARVO Resolution on the Use of Animals in Research. A baseline examination of all eyes was conducted prior to injection in order to determine ocular health. All eyes presented with mild diffuse fluorescein staining, characteristically seen in the normal rabbit eye. The health of all eyes fell within normal limits. Rabbits weighing between 2.5 and 3.0 kg were anesthetized by intramuscular injection of 0.5-0.7 mL/kg rodent cocktail (100 mg/mL ketamine, 20 mg/mL xylazine, and 10 mg/mL acepromazine). One drop of proparacaine hydrochloride (0.5% Ophthaine, Bristol-Myers Squibb) was applied to the eye prior to injection. Twenty microliters of bacterial suspension (1×10² CFU) prepared as described above was injected into the central corneal stroma of a randomly assigned eye while the other eye remained naive. Injections, simulating perforation of the corneal epithelium, were performed using a 30-gauge 112-inch needle and a 100 μL syringe.

For the first series of experiments, a 5day dosing regimen of BPI protein product (test drug) was as follows: on Day 0 of the study, 40 μL of test drug or vehicle control was delivered to the test eye at 2 hours (-2) and 1 hour (−1) prior to intrastromal bacterial injection (time 0), then at each of the following 10 hours (0 through +9 hrs) post-injection for a total of 1-2 doses (40 μL/dose); on each of Days 14 of the study, 40 μL of test drug or vehicle control was delivered to the test eye at each of 10 hours (given at the same time each day, e.g., 8am-5pm). For Expt. 1, 9 animals were treated, 5 with rBPI₄₂ (1 mg/mL in S mM citrate, 150 mM NaCl, 0.1% poloxamer 188, 0.002% polysorbate 80) and 4 with buffered vehicle (5 mM citrate, 150 mM NaCl, 0.2% poloxamer 188, 0.002% polysorbate 80). For Expt. 2, 10 animals were treated, 5 with rBPI₂₁ (2 mg/mL in 5 mM citrate, 150 mM NaCl, 0.2% poloxamer 188, 0.002% polysorbate 80) and 5 with buffered vehicle. For each of Expt. 3 and Expt. 4, 5 animals were treated with XMP.112 (1 mg/mL in 150 mM NaCl) and XMP.105 (1 mg/mL in 150 mM NaCl), respectively, and 5 animals with buffered vehicle. For Expt. 5, 5 animals were treated with rBPI₂₁ (2 mg/mL in 5 mM citrate, 150 mM NaCl, 0.2% poloxamer 403, 0.002% polysorbate 80) and 5 animals with placebo (5 mM citrate, 150 mM NaCl, 0.2% poloxamer 403, 0.002% polysorbate 80).

For these experiments, eye examinations were conducted two times each day for each 5-day study via slit lamp biomicroscopy to note clinical manifestations. Conjunctival hyperemia, chemosis and tearing, mucous discharge were graded. The grading scale for hyperemia was: 0 (none); 1 (mild); 2 (moderate); and 3 (severe). The scale for grading chemosis was: 0 (none); 1 (visible in slit lamp); 2 (moderate separation); and 3 (severe ballooning). The scale for grading mucous discharge was: 0 (none) 1 slight accumulation); 2 (thickened discharge); and 3 (discrete strands). Photophobia was recorded as present or absent. Tearing was recorded as present or absent. The corneal ulcer, when present, was assessed with respect to height (mm), width (mm), and depth (% of corneal thickness). Neovascularization was graphed with respect to the affected corneal meridians. Photodocumentation was performed daily as symptoms progressed throughout the experimental procedure.

At the completion of the 5-day study period, all rabbits were sacrificed via a lethal dose of sodium pentobarbital (6 grs/mL). Corneas were harvested and fixed in half-strength Karnovsky's fixative. The corneas were processed for light microscopy using Gram stain to assay for the presence of microbial organisms and using hematoxylin and eosin to assay for cellular infiltrate.

Examinations were conducted after injection of Pseudomonas at 4, 24, 28, 48, 52, 72, 76, and 96 hours for these experiments. Additional examinations were conducted at 100 and 168 hours for Expt 3 with XMP.112 since neovascularization progressed more slowly in this experiment than it did in others. The results of these examinations are reported in Table 1 for Expt. 5 wherein the BPI protein product tested (rBPI₂₁, in a formulation with poloxamer 403) provided the most potent effects.

TABLE 1
Summary of Clinial Observations

| | | | | | | | Neovas- | | Ulcer Size | |
|---|---|---|---|---|---|---|---|---|---|---|
| | Hyperemia* | | Chemosis* | | Mucous* | | cularization | | (mm) | |
| Examination | rBPI₂₁ | Plbo. | rBPI₂₁ | Plbo. | rBPI₂₁ | Plbo. | rBPI₂₁ | Plbo. | rBPI₂₁ | Plbo. |
| Exam 1 | 1.2 | 1.0 | 0.2 | 0.3 | 0.5 | 0 | None | None | 1 ulcer | 1.4 |
| 4 hours | | | | | | | | | 2 mm | |
| Exam 2 | 0.9 | 1.6 | 0.2 | 1.0 | 0.3 | 0.5 | None | None | 1 ulcer | 3.4 |
| 24 hours | | | | | | | | | 6 mm | |
| Exam 3 | 0.6 | 1.7 | 0.2 | 1.1 | 0.6 | 1.3 | None | None | 1 ulcer | 5.2 |
| 28 hours | | | | | | | | | 7 mm | |
| Exam 4 | 0.6 | 2.4 | 0.2 | 1.3 | 0.4 | 2.1 | None | None | 1 ulcer | 11.4 |
| 48 hours | | | | | | | | | 12 mm | 3 melt |
| | | | | | | | | | 1 melt | 1 thinning |
| Exam 5 | 0.8 | 2.4 | 0.2 | 1.2 | 0.2 | 1.6 | None | Yes | 1 ulcer | 11.4 |
| 52 hours | | | | | | | | (1/5) | 12 mm | 3 melt |
| | | | | | | | | | 1 melt | 1 thinning |
| Exam 6 | 0.6 | 2.4 | 0 | 0.8 | 0.2 | 1.0 | None | Yes | 1 ulcer | 11.4 |
| 72 hours | | | | | | | | (1/5) | 12 mm | 4 melt |
| | | | | | | | | | melt & thin | 1 thinning |
| Exam 7 | 0.6 | 2.4 | 0 | 0.2 | 0.2 | 0.8 | None | Yes | 1 ulcer | 11.4 |
| 76 hours | | | | | | | | (2/5) | 12 mm | 4 melt |
| | | | | | | | | | melt & thin | 3 thinning |
| Exam 8 | 0.6 | 2.4 | 0 | 0.2 | 0.2 | 0.8 | None | Yes | 1 ulcer | 11.4 |
| 96 hours | | | | | | | | (2/5) | 12 mm | 4 melt |
| | | | | | | | | | melt & thin | 3 thinning |

*Mean scores of clinical observations graded on a scale of 0 (none) to 3 (severe).

The results set out in Table 1 reveal that treatment of the eye prior to and after perforation injury and injection of Pseudomonas provided substantial benefits in terms of reduced hyperemia, chemosis and mucous formation, as well as reduction in incidence of neovascularization along with reduced incidence and severity of corneal ulceration. At four hours after Pseudomonas injection, fluorescein staining of the cornea in both treated and control animals revealed small areas of staining consistent with the injection (puncture) injury. At 28 hours after injection, the rBPI₂₁ treated eye evidenced clear ocular surfaces and typically were free of evidence of hyperemia, chemosis and mucous discharge while the vehicle treated eyes showed clouding of the ocular surface resulting from corneal edema and infiltration of white cells. Iritis was conspicuous in the vehicle treated eyes at 28 hours after injection and fluorescein dye application typically revealed areas of devitalized epithelium; severe hyperemia and moderate to severe chemosis and mucous discharge were additionally noted. At 48 hours after injection, mild hyperemia was sometimes noted in the rBPI₂₁ treated eyes but mucous discharge and chemosis were absent; the rBPI₂₁ treated corneas were otherwise typically clear and healthy appearing, as evidenced by the application of fluorescein dye. Vehicle treated eyes at 48 hours post infection displayed severe hyperemia, chemosis and mucous discharge were present; some corneas displayed corneal melting and thinning along with an ulcerating area clouded as a result of edema, cellular infiltration and fibrin deposition. At 52 hours following injection, rBPI₂₁ treated eyes exhibited clear and healthy corneas which resisted staining with fluorescein, indicating that the formulation is safe and non-toxic to the corneal epithelium. In vehicle treated eyes at 52 hours post infection, sloughing of corneal epithelium was evident and while chemosis was decreasing, hyperemia was severe. In these experiments, several vehicle treated eyes presented with neovascularization, with vessels growing inward toward the central cornea. This manifestation was not noted in any rBPI21 treated eye.

Pathohistological evaluation of the rBPI₂₁ treated corneas stained with hematoxylin and eosin revealed healthy, intact corneal epithelium and stroma; the tissue was free of white cell infiltration. In contrast, evaluation of the vehicle treated corneas revealed absence of an epithelium and extensive infiltration of white cells into the corneal stroma.

Additional pathohistological evaluation of the rBPI₂₁ treated corneas stained with toluidine blue also revealed healthy, intact corneal epithelium and stoma, and further revealed corneal tissue free of Pseudomonas organisms. In contrast, evaluation of the vehicle treated corneas revealed rod shaped Pseudomonas organisms in the tissue and the presence of white cells advancing toward the organisms in the tissue. These results indicate effective corneal penetration of the rBPI₂₁ and effective sterilization of the tissue without neovascularization.

FIGS. 1 and 2 respectively provide a photographic comparison of representative control (placebo) and treated (rBPI₂₁/poloxamer 403) results at 72 hours. The fluorescein stained treated eye (FIG. 2) is healthy and clear; no keratitis is evident, confirming safety of chronic use in rabbits. In the “control” eye shown, the perithelium has severely melted; the thinning central cornea is ready to perforate. Severe hyperemia and moderate mucous discharge is apparent. Chemosis was not evident.

The rBPI₂₁ formulation with poloxamer 403 tested in these experiments achieved the most dramatic beneficial antimicrobial and anti-angiogenic effects when compared with those of the other BPI protein product formulations tested in this severe Pseudomonas injury/infection rabbit model. Benefits in terms of suppression of neovascularization were noted for treatment with the rBPI₄₂, rBPI₂₁ (with poloxamer 188) and XMP.112 preparations whereas treatment with XMP.105 resulted in one of the five treated eyes showing neovascularization as opposed to none for the vehicle treated animals. Further, no significant effects in reduction of hyperemia, chemosis, mucous formation and tearing were noted. The contrast in efficacy of the BPI₂₁/poloxamer 403 results (Expt. 5) with the lesser efficacy of the other products and formulations in that study suggested that formulation components, dosage and dosage regimen for a particular BPI protein product may all have a significant role in optimizing beneficial effects associated with practice of the invention.

The following Example illustrates practice of routine procedures designed to assess, in part, effects of formulation components and dosage regimens on optimization of beneficial effects attending practice of the present invention.

### EXAMPLE 2

### Effect of BPI Protein Product Formulations and Dosing on Pseudomonas Infection in a Corneal Ulceration Rabbit Model

The effect of BPI protein product administration following Pseudomonas infection was evaluated in a corneal infection/ulceration rabbit model using rBPI₂₁ in various formulations with (A) poloxamer 188, (B) poloxamer 333, and (C) poloxamer 403 (as in Expt. 5 of Example 1).

For these experiments, the infectious organism was a strain of Pseudomonas aeruginosa 19660 prepared and used to inject rabbits as described in Example 1. In a first set of studies, no beneficial effects were observed when the test product dosing regimen included no pre-injection doses of BPI protein product and treatment was withheld until commencement of ulcer formation at about 12-16 hours after the bacterial injection. Briefly put, the dosing regimen of BPI protein product employed was not sufficient to overcome the massive destructive effects of large numbers of microorganisms, where the infection was allowed to develop for 12-16 hours before intervention.

In a second variant dosing and formulation study, the dosing regimen was as described in Example 1 except that animals were not dosed at 2 hours and 1 hour prior to injection with Pseudononas, but were dosed at the time of injection and then each hour for 12 hours on the first day of the 5 day experiment. Treatment was as in Example 1 for days 2-5. For these experiments, animals were treated as follows: 5 with rBPI₂₁ formulated with poloxamer 188 (formulation A: 2 mg/mL rBPI₂₁ in 5 mM citrate, 150 mM NaCl, 0.2% poloxamer 188, 0.002% polysorbate 80), 5 with rBPI₂₁ formulated with poloxamer 333 (formulation B: 2 mg/mL rBPI₂₁ in 5 mM citrate, 150 mM NaCl, 0.2% poloxamer 333, 0.002 polysorbate 80), 5 with rBPI₂₁ formulated with poloxamer 403 (formulation C: 2 mg/mL rBPI₂₁ in 5 mM citrate, 150 mM NaCl, 0.2% poloxamer 403, 0.002% polysorbate 80) and S with phosphate buffered saline (PBS) control. Eye examinations were carried out as described in Example 1 and the animals sacrificed at the end of the 5 day protocol.

Formulation C treated eyes exhibited less hyperemia than saline treated eyes up to the 28 hour evaluation. The effect was less at the 28 hour evaluation, while subsequent hyperemia scores were equivalent between test and control groups. Formulation C also consistently presented lower hyperemia scores than formulation A and B, suggesting that eyes treated with formulation C were not eliciting as much of an inflammatory response as observed the eyes in the other treated groups.

Formulation C also elicited significantly lower scores for chemosis than control at the 28 hour evaluation. This effect was less at the 24 hour evaluation. Clinical scores for chemosis were consistently lower for group C than any of the other treated groups. As hyperemia increases, the vessels become progressively permeable, allowing increased serum deposition into the tissues. The formulation C treated eyes, which elicited the lowest degree of hyperemia, presented the lowest degree of chemosis.

During the first 28 hours of the study, formulation C treated eyes presented consistently lower mucous discharge scores than all other groups. Neutrophil containing mucous is generally produced in response to irritation. Control treated eyes produced markedly greater mucous discharge during the first 28 hours of the study than any of the active treated groups, indicating a high degree of distress.

Formulation C treated eyes displayed the smallest ulcers during the first 28 hours of the study, and in accordance with the other clinical data, was the most effective antimicrobial agent of the three formulations tested. Formulation B achieved beneficial results superior to formulation A with respect to bactericidal capability, although the differences were less than that between formulations A and C. All eyes, however, were overwhelmed by the Pseudomonas over the 28 to 48 hour period.

In these experiments, formulation C demonstrated potent antimicrobial properties and was able to suppress ulcer progression.

### EXAMPLE 3

### Effect of Administration of BPI Protein Product and Antibiotic for Pseudomonas Infection in a Corneal Ulceration Rabbit Model

The effect of BPI protein product administration for Pseudomonas infection is evaluated in a corneal infection/ulceration rabbit model using a BPI protein product, such as rBPI₂₁, in various formulations alone and in co-administration with various antibiotics. Experiments are performed as described in Examples 1 and 2, but wherein the BPI protein product is administered as an adjunct to antibiotic treatment. Experiments are performed as described in Examples 1 and 2, except that antibiotic dosing is performed in additional to dosing with BPI protein product. For these experiments, the antibiotic dose is administered before, simultaneously with, or after each dose of BPI protein product.

Numerous modifications and variations of the above-described invention are expected to occur to those of skill in the art. Accordingly, only such limitations as appear in the appended claims should be placed thereon.

What is claimed is:

1. A method for treating corneal epithelium injury associated infection comprising topically administering to the cornea of a subject having a corneal epithelium injury a bactericidal/permeability-increasing (BPI) protein product in an amount effective to reduce hyperemia, chemosis, mucous discharge, neovascularization or ulcer formation.

2. The method of claim 1 wherein the BPI protein product is an amino-terminal fragment of BPI protein.

3. The method of claim 1 wherein the BPI protein product is rBPL₂₁.

4. The method of claim 1 wherein the BPI protein product is rBPI₂₃.

5. The method of claim 1 wherein the BPI protein product is rBPI₄₂.

6. The method of claim 1 further comprising administration of a non-BPI antibiotic or a non-BPI anti-fungal agent.

7. The method of claim 1 further comprising administration of an anti-inflammatory agent.
